(19) United States
(12) Patent Application Publication
(10) Pub. No.: US 2025/0295692 A1
(43) Pub. Date: Sep. 25, 2025

(54) ISOLATION OF ANTI-INFLAMMATORY NEUTROPHIL OR MONOCYTE SUBSET AND USE IN TREATING CARDIOVASCULAR DISEASES

(71) Applicant: Virginia Tech Intellectual Properties, Inc., Blacksburg, VA (US)

(72) Inventors: Liwu Li, Blacksburg, VA (US); Shuo Geng, Blacksburg, VA (US)

(21) Appl. No.: 19/088,065

(22) Filed: Mar. 24, 2025

Related U.S. Application Data
(60) Provisional application No. 63/568,634, filed on Mar. 22, 2024.

(51) Int. Cl. A61K 35/15; A61K 40/10; A61K 40/42; A61P 29/00; A61P 37/06; C07K 14/705; C12N 5/00; C12N 5/0787; C12N 9/22; C12N 15/11; C12N 15/113

(52) U.S. Cl. CPC A61K 35/15; A61K 40/10; A61K 40/4224; A61P 29/00; A61P 37/06; C07K 14/7056; C07K 14/70596; C12N 5/0018; C12N 5/0642; C12N 9/22; C12N 15/111; C12N 15/113; C12N 2310/11; C12N 2310/14; C12N 2310/20; C12N 2501/22; C12N 2510/00

(57) ABSTRACT

Provided are neutrophils and monocytes having unique combinations of cell surface markers capable of enhancing TRAM^{lo} neutrophil or monocyte subsets, or capable of reprogramming neutrophils and monocytes into TRAM deficient neutrophils and monocytes, including methods of isolating the neutrophils and monocytes for use in treating chronic inflammatory diseases. The enhanced or reprogramed neutrophils may express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof, and the enhanced or reprogrammed monocytes may express surface markers comprising CD14⁺, CD16⁺, and at least one of CD24^{hi}, CD200R^{hi}, or CD84^{hi}.

### CROSS-REFERENCE TO RELATED APPLICATIONS

This application claims priority and is related to U.S. Provisional Application Ser. No. 63/568,634 filed on Mar. 22, 2024, and entitled “Isolation of Anti-Inflammatory Neutrophil or Monocyte Subset and Use in Treating Cardiovascular Diseases”, which is hereby incorporated herein by reference in its entirety.

### GOVERNMENT SUPPORT CLAUSE

This invention was made with government support under NIH R01 AI 136386 awarded by the National Institutes of Health (NIH). The government has certain rights in the invention.

### TECHNICAL FIELD

This disclosure relates to neutrophils and monocytes with unique combinations of cell surface markers capable of enhancing TRAM^{lo} neutrophil or monocyte subsets, or reprogramming neutrophils and monocytes into TRAM deficient neutrophils and monocytes, and more particularly to methods of isolating the neutrophils and monocytes for use in treating chronic inflammatory diseases.

### BACKGROUND

Despite extensive studies, atherosclerosis and related cardiovascular complications still remain as the leading cause of morbidity and mortality worldwide. Although mechanistically less studied, there is a compelling phenotypic connection between elevated circulating neutrophil counts with the development of atherosclerosis in both experimental animals and human patients. Neutrophils account for around 50-70% of human circulating leukocytes, and in human atherosclerosis, high circulating neutrophil ratios have been tightly associated with characteristics of rupture-prone atherosclerotic lesions. In experimental animals, neutrophilia—promoted by hyperlipidemia—accelerates early atherosclerosis. Neutrophils contribute to atherosclerosis pathogenesis through enhanced adhesion to vasculatures and subsequent damage to endothelium via production of adhesion/swarming molecules, such as leukotriene B4 (LTB4) and ICAM-1, as well as tissue degrading molecules, such as myeloperoxidase (MPO) and elastase. Therefore, depletion of inflammatory neutrophils has been shown to reduce the atherosclerotic lesion burden.

Paradoxically, neutrophils are also beneficial during the resolution of inflammation through expressing resolving mediators such as resolvin D1 (RvD1). Recent clinical studies reveal that the ratios of LTB4 versus RvD1 closely correlate with clinical severity of cardiovascular diseases in human patients. However, the mechanisms underlying the paradoxical roles of neutrophils in promoting or resolving inflammation related to atherosclerosis pathogenesis and treatment are not well-understood.

Systemic dysregulations of neutrophils are increasingly recognized as risk factors during atherosclerosis pathogenesis, and efforts in restoring neutrophil homeostasis may be critical for the effective treatment of atherosclerosis. However, effective approaches in the generation of resolving neutrophils amenable for reducing atherosclerosis pathogenesis are not readily available, due to the limited understanding of complex mechanisms responsible for neutrophil polarization dynamics. Further, although conventional therapies against atherosclerosis based on small chemicals or biologic molecules have been widely developed, there are intrinsic caveats associated with these approaches as reflected in systemic side-effects and limited delivery efficacy.

Therefore, there remains a need for novel leukocyte therapies and methods of isolating such leukocyte therapies for use in treating chronic inflammatory diseases and disorders.

### SUMMARY

Provided are various methods directed to enriching and reprogramming leukocytes, such as neutrophils and monocytes, for isolating a subset of the leukocytes with anti-inflammatory functions capable of reducing inflammation. Beneficially, the enriched or reprogrammed leukocytes may be used in methods of treating inflammatory diseases and disorders as further described herein.

In one example, a method of enriching TRAM^{lo} neutrophils, or reprogramming innate neutrophils into TRAM deficient neutrophils comprises receiving innate neutrophils isolated from a heterologous or allogenic donor, and sorting the innate neutrophils to result in TRAM^{lo} neutrophils or modifying the innate neutrophils to result in TRAM deficient neutrophils, wherein the TRAM^{lo} neutrophils or TRAM deficient neutrophils express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof.

In a further example, a method of enriching TRAM^{lo} monocytes, or reprogramming human monocytes into TRAM deficient monocytes comprises receiving human monocytes isolated from a heterologous or allogenic donor, and sorting TRAM^{lo} monocytes comprising TRAM^{lo} intermediate monocytes or TRAM^{lo} non-canonical monocytes, or modifying the human monocytes to result in TRAM deficient monocytes, wherein the TRAM^{lo} monocytes or the TRAM deficient monocytes express surface markers comprising CD14⁺, CD16⁺, and at least one of CD24^{hi}, CD200R^{hi}, or CD84^{hi}.

In a further example, a method of treating a chronic inflammatory disease in a subject comprises administering TRAM^{lo} neutrophils or TRAM deficient neutrophils to the subject, wherein the TRAM^{lo} neutrophils or TRAM deficient neutrophils express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof.

In a further example, a method of treating a chronic inflammatory disease in a subject comprises administering TRAM^{lo} monocytes or TRAM deficient monocytes to the subject, wherein the TRAM^{lo} monocytes or TRAM deficient monocytes express surface markers comprising CD14⁺, CD16⁺, and at least one of CD24^{hi}, CD200R^{hi}, or CD84^{hi}.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF DRAWINGS

FIG. 1. Transfusion of Tram^{-/-} neutrophils ameliorates atherosclerosis: Male ApoE^{-/-} Tram^{+/+} recipient mice were first fed with HFD for 4 weeks. Neutrophils isolated from ApoE^{-/-} Tram^{+/+} mice or Apoe^{-/-} Tram^{-/-} mice were adoptively transferred by intravenous injection to HFD-fed recipient mice once a week for an additional 4 weeks. Samples were collected 1 week after the last neutrophil transfer. FIG. 1A shows representative images of H&E stained atherosclerotic lesions and quantification of plaque size demonstrated as the percentage of lesion area within aortic root area. Scale bar, 300 μm. FIG. 1B shows representative images of Oil Red O stained atherosclerotic plaques and quantification of lipid deposition within lesion area. Scale bar, 300 μm. FIG. 1C shows representative images of Picrosirius red stained atherosclerotic plaques and quantification of collagen content within lesion area. Scale bar, 100 m. FIG. 1D shows the determination of plasma RevD1, elastase, and LTB4 levels by ELISA. Data are presented as means±SEM. *P<0.05, **P<0.01, and ***P<0.001; Student's 2-tailed t test (n=10 for each group).

FIG. 2. Tram^{-/-} neutrophils exert protective effects on endothelial cells: (A-C) ApoE^{-/-} Tram^{+/+} recipient mice were first fed with HFD for 4 weeks. Neutrophils isolated from ApoE^{-/-} Tram^{+/+} mice or Apoe^{-/-} Tram^{-/-} mice were adoptively transferred by intravenous injection to HFD-fed recipient mice once a week for an additional 4 weeks. FIG. 2A shows results one week after the final cell transfer, Evans Blue solution was intravenously injected, and Evans Blue leaked into the aorta was analyzed (n=4). FIG. 2B shows VCAM-1 expression on aortic endothelial cells was analyzed by FACS. Representative FACS histogram and quantification are displayed (n=5). FIG. 2C shows FACS analysis of Ly6C⁺⁺ pro-inflammatory monocyte frequency in the aorta (n=5). FIG. 2D shows WT or Tram^{-/-} murine neutrophils were co-cultured with the monolayer of bend.3 cells in a Transwell system for 24 hours. Evans Blue leakage to the lower chambers was determined by the absorbance at 620 nm (n=5). FIG. 2E shows WT or Tram^{-/-} murine neutrophils were co-cultured with the monolayer of MCECs in a Transwell system for 24 hours. Evans Blue leakage to the lower chambers was determined by the absorbance at 620 nm (n=5). FIG. 2F shows WT or Tram^{-/-} murine neutrophils were co-cultured with MCECs for 24 hours, and VCAM-1 expression on MCECs was analyzed by FACS. Representative FACS histogram and quantification are displayed (n=5). FIG. 2G shows WT or Tram^{-/-} murine neutrophils were co-cultured with MCECs for 24 hours, and the death on MCECs was determined by PI staining. Representative FACS histogram and quantification are displayed (n=5). Data are presented as means±SEM. *P<0.05, **P<0.01, and ***P<0.001; Student's 2-tailed t test.

FIG. 3. TRAM deletion reduces FLAP expression and impedes the nuclear distribution of LOX5 in neutrophils: WT and Tram^{-/-} murine neutrophils were treated with oxLDL (10 μg/mL) or cholesterol (10 μg/mL) for 48 hours. FIG. 3A shows secretion of RevD1 by neutrophils was quantified by ELISA (n=4). FIG. 3B shows protein level of FLAP was examined by Western blotting, and relative expression was normalized to β-actin (n=3). FIG. 3C confocal microscopy imaging of the subcellular distribution of LOX5 in neutrophils. Scale bar, 5 μm. FIG. 3D shows confocal microscopy imaging of the subcellular distribution of TRAM in neutrophils. Scale bar, 5 μm. Data are presented as means±SEM. *P<0.05, **P<0.01, and ***P<0.001; one-way ANOVA.

FIG. 4. TRAM deletion abrogates the molecular machineries responsible for elastase production: WT and Tram^{-/-} murine neutrophils were treated with oxLDL (10 μg/mL) for 48 hours. FIG. 4A shows secretion of elastase by neutrophils was quantified by ELISA (n=5). FIG. 4B shows neutrophils labeled with CellROX, and ROS levels quantified by FACS (n=5). FIG. 4C shows neutrophils stained with anti-PMP70 and anti-LAMP1 antibodies, and the localization of peroxisomes and lysosomes examined by confocal microcopy. Scale bar, 5 μm.

FIG. 4D shows confocal microscopy imaging of the subcellular distribution of PMP70⁺ peroxisomes and CAMKII in neutrophils. Scale bar, 5 μm. FIG. 4E shows confocal microscopy imaging of the subcellular distribution of PMP70⁺ peroxisomes and SYK in neutrophils. Scale bar, 5 μm. FIG. 4F shows protein level of SESN1 examined by Western blotting, and relative expression normalized to j-actin (n=3). Data are presented as means±SEM. *P<0.05, **P<0.01, and ***P<0.001; one-way ANOVA.

FIG. 5. Human CD177¹⁰ neutrophils exhibit similar resolving characteristics to mouse Tram^{-/-} neutrophils: FIG. 5A shows FACS analysis of TRAM⁺ population frequency in CD66b^{lo}+CD177^{lo} and CD66b⁺ CD177^{hi} neutrophils from the peripheral blood of healthy individuals (n=4). FIG. 5B shows CD177^{lo} and CD177^{hi} neutrophils purified from the peripheral blood of healthy individuals. CD177^{lo} neutrophils were labeled with Hoechst 33342, and CD177^{hi} neutrophils were labeled with DRAQ5. The swarming of the neutrophils were recorded and quantified (n=4). FIG. 5C and FIG. 5D show purified CD177^{lo} and CD177^{hi} neutrophils stimulated with or without fMLP (100 nM) for 4 hours. The secretion of LTB4 (FIG. 5C) and elastase (FIG. 5D) were analyzed by ELISA (n=5). Data are presented as means±SEM. *P<0.05, and **P<0.01; Student's 2-tailed t test.

FIG. 6 demonstrates a bubble plot identifying differentially expressed genes after a single cell RNA sequencing (scRNAseq) analysis of murine neutrophils, revealing the nature of anti-inflammatory, resolving neutrophils capable of reducing inflammation and improving tissue homeostasis/function. Purified naïve mouse bone marrow cultured in the presence of G-CSF (100 ng/ml) were used to perform single cell sequencing.

FIG. 7 Transfusion of Tram−/− neutrophils ameliorates atherosclerosis: Female ApoE−/− Tram+/+ recipient mice were first fed with HFD for 4 weeks. Neutrophils isolated from ApoE−/− Tram+/+ mice or Apoe−/− Tram−/− mice were adoptively transferred by intravenous injection to HFD-fed recipient mice once a week for an additional 4 weeks. Samples were collected 1 week after the last neutrophil transfer. FIG. 7A shows representative images of H&E stained atherosclerotic lesions and quantification of plaque size demonstrated as the percentage of lesion area within aortic root area. Scale bar, 300 μm. FIG. 7B shows representative images of Oil Red O stained atherosclerotic plaques and quantification of lipid deposition within lesion area. Scale bar, 300 μm. FIG. 7C shows representative images of Picrosirius red stained atherosclerotic plaques and quantification of collagen content within lesion area. Scale bar, 100 μm. Data are presented as means±SEM. **P<0.01, and ***P<0.001; Student's 2-tailed t test.

FIG. 8 Transfusion of Tram−/− neutrophils decreases circulating MPO level: ApoE−/− Tram+/+ recipient mice were first fed with HFD for 4 weeks. Neutrophils isolated from ApoE−/− Tram+/+ mice or Apoe−/− Tram−/− mice were adoptively transferred by intravenous injection to HFD-fed recipient mice once a week for an additional 4 weeks. Plasma samples were collected 1 week after the last neutrophil transfer, and MPO level was assessed by ELISA. Data are presented as means±SEM. **P<0.01; Student's 2-tailed t test.

FIG. 9 Tram−/− Neutrophils reduce surface expression of VCAM-1 on endothelial cells: FIG. 9A shows graphs for data WT or Tram−/− murine neutrophils co-cultured with bEnd.3 cells for 24 hours, and VCAM-1 expression on bEnd.3 cells analyzed by FACS. Representative FACS histogram and quantification are displayed (n=3). FIG. 9B shows graphs for data WT or Tram−/− murine neutrophils co-cultured with HUVECs for 24 hours, and VCAM-1 expression on HUVECs analyzed by FACS. Representative FACS histogram and quantification are displayed (n=3). Data are presented as means±SEM. **P<0.01; Student's 2-tailed t test.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular neutrophil or monocyte compositions and methods of use or isolation, which can vary and are understood by skilled artisans. It is further to be understood that all terminology used herein is for the purpose of describing particular embodiments only and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4% This applies regardless of the breadth of the range.

The term “about,” as used herein, refers to variation in the numerical quantity that can occur, for example, through typical measuring techniques and equipment, with respect to any quantifiable variable, including, but not limited to, mass, volume, temperature, and time. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “control” can refer to an alternative subject or sample used in an experiment for comparison purpose and included to minimize or distinguish the effect of variables other than an independent variable. A “suitable control” is one that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a reprogrammed neutrophil as described herein that can be effective to enhance anti-tumor immune responses.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long non-coding RNA and shRNA.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, “pharmaceutical formulation” or “pharmaceutical composition” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, nontoxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “preventative,” “prevent,” or “preventing” refers to hindering or stopping a disease or condition before it occurs, even if undiagnosed, or while the disease or condition is still in the sub-clinical phase.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect. The therapeutic effect can be treating and/or preventing non-resolving cancer and/or related diseases or conditions.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

The present disclosure describes methods of enriching TRAM^{lo} neutrophils or TRAM^{lo} monocytes for the treatment of chronic inflammatory diseases. In further embodiments, the methods reprogram innate neutrophils or human monocytes into TRAM deficient neutrophils or TRAM deficient monocytes conducive for the treatment of chronic inflammatory diseases. In some embodiments, the disclosed neutrophils and monocytes can be used to treat and/or prevent a chronic inflammatory disease in a subject in need thereof. The present disclosure provides for a subset of leukocytes, such as neutrophils and monocytes, that are capable of improving vasculature integrity and reducing atherosclerosis in a subject. In some aspects, the innate leukocytes such as the neutrophils and monocytes, possess a minor homeostatic resolving subset characterized by a reduced expression of inflammatory mediators and an elevated expression of anti-inflammatory resolving mediators, which are correlated with a reduced expression of the inflammatory signaling adaptor, TRAM.

In some aspects, innate immune leukocytes such as neutrophils and monocytes are naturally equipped with tissue tropic capabilities to effectively survey and home into inflamed areas. Emerging studies have attempted the utilization of neutrophil-based approaches in delivering therapeutic compounds to treat diseases, however, neutrophils are known to exhibit diverse inflammatory characteristics, including, for example, degranulation and release of inflammatory mediators as well as swarming and aggregation with neighboring cells and tissues. These tissue toxic effects of neutrophils hinder the proper development of neutrophil-based delivery or cell therapies. In some aspects, the present disclosure has identified a unique subset of neutrophils and monocytes with potent homeostatic resolving capabilities, enabled by TRAM reduction.

TRAM (also named TICAM-2) is an adaptor protein for toll-like receptors (TLRs). In some aspects, as discovered in the present disclosure, TRAM, expressed by neutrophils and monocytes, can mediate atherosclerotic stress and the inflammatory effects of lipids. In aspects, oxidized low-density lipoprotein (oxLDL) or cholesterol can cause generic membrane stress, where TRAM may serve as a general membrane stress sensor for oxLDL or free cholesterol in addition to TLR ligands. In some aspects, TRAM is one of the few innate membrane adaptors with lipid anchors to a stressed membrane region. In further implementations, and without being limited to any particular mechanism or theory, TRAM can contribute to inflammatory and apoptotic neutrophil polarization through mediating peroxisome-mediated activation of SYK and CAMKII, leading to the induction of LOX5AP. Nuclear localization of LOX5 controlled by LOX5AP enables the production of inflammatory mediators, such as LTB4, whereas cytosolic LOX5 participates in the generation of anti-inflammatory or resolving mediators, such as RvD1. TRAM-mediated inflammatory signaling also can trigger inflammasome activation and degranulation which lead to enhanced release of tissue-damaging elastase. Elastase can play a crucial role in the pathogenesis of atherosclerosis, with leukocytes in atherosclerotic lesions exhibiting a high expression of elastase. In some aspects, elevated plasma levels of elastase can induce endothelial damage, which is an early cause of atherosclerosis initiation. Therefore, the present disclosure identifies the need for TRAM^{lo} or TRAM deficient neutrophils and monocytes, as TRAM can serve as a key switch, toggling the generation of either pro-inflammatory or resolving neutrophils and monocytes.

In some embodiments, TRAM can serve as a generic stress sensor for diverse stress signals, including, but not limited to oxidized phosphor-lipids. TRAM is a unique signaling adaptor with a covalently conjugated lipid moiety, enabling TRAM to sense membrane stress mediated by lipid-raft formation. In some examples, both oxLDL and free cholesterol can increase membrane stress and facilitate lipid-raft formation. Therefore, in some aspects, TRAM can serve as a general stress sensor, including oxLDL, where oxLDL can potently induce the membrane clustering of TRAM on neutrophils and monocytes.

In some embodiments, a method of TRAM deficient neutrophils are reprogrammed into the resolving state, and can potently improve vascular integrity and suppress atherosclerosis pathogenesis when adoptively transfused into a subject with a chronic inflammatory disease or disorder.

In some embodiments, a method of enriching TRAM^{lo} neutrophils, or reprogramming innate neutrophils into TRAM deficient neutrophils are provided. In embodiments, the method comprises receiving innate neutrophils isolated from a heterologous or allogenic (homologous) donor. In some aspects, the innate neutrophils are isolated from human blood, including, for example, human peripheral blood. In further aspects, the neutrophils may be derived from stem cell culture. In further aspects, the innate neutrophils may be sorted to result in TRAM^{lo} neutrophils or modified to result in TRAM deficient neutrophils. The sorted TRAM^{lo} neutrophils or modified TRAM deficient neutrophils may be optionally enriched and further used in methods of treating a chronic inflammatory disease in a subject, as will be further described herein.

In embodiments, the TRAM^{lo} neutrophils or TRAM deficient neutrophils may be sorted or modified based on the surface markers expressed. In some aspects, the TRAM^{lo} neutrophils or TRAM deficient neutrophils may express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof. In further embodiments, the TRAM neutrophils or TRAM deficient neutrophils may express surface markers comprising CD177^{lo} and Dectin 2 (Clec4n)^{lo}, or wherein the surface markers comprise CD177^{lo}, CD84^{hi}, Ly6E/Ly6A^{hi}, CD24^{hi}, and EHD1^{lo}. In some aspects, the TRAM^{lo} neutrophils or TRAM deficient neutrophils exhibit reduced expression of inflammatory mediators comprising LTB4, elastase, MPO, CD11b, CD49d, or a combination thereof. In further aspects, the TRAM^{lo} neutrophils or TRAM deficient neutrophils exhibit elevated expression of resolving mediators comprising RvD1, CD200R, CD24, CD84, Sestrin1 (SESN1), Sestrin3 (SESN3), CD84, Ly6E/Ly6A, MCL1, or a combination thereof. The elevated expression of anti-inflammatory resolving mediators paired with a reduced expression of inflammatory mediators can beneficially provide for compositions and methods for use in treating chronic inflammatory diseases.

In further embodiments, a method of enriching TRAM^{lo} monocytes, or reprogramming human monocytes into TRAM deficient monocytes are provided. In embodiments, the method comprises receiving human monocytes isolated from a heterologous or allogenic (homologous) donor. In some aspects, the innate neutrophils are isolated from human blood, including, for example, human peripheral blood. In further aspects, the monocytes may be derived from stem cell culture. In further aspects, the human neutrophils may be sorted to result in TRAM^{lo} monocytes or modified to result in TRAM deficient monocytes. The sorted TRAM^{lo} monocytes or modified TRAM deficient monocytes may be optionally enriched and further used in methods of treating a chronic inflammatory disease in a subject, as will be further described herein.

In embodiments, the TRAM^{lo} monocytes or TRAM deficient monocytes may be sorted or modified based on the surface markers expressed. In some aspects, the TRAM^{lo} monocytes or TRAM deficient monocytes may express surface markers comprising CD14⁺, CD16⁺, and at least one of CD24^{hi}, CD200R^{hi}, or CD84^{hi}. In some aspects, the TRAM^{lo} monocytes or TRAM deficient monocytes exhibit reduced expression of inflammatory mediators comprising LTB4, elastase, MPO, CD11b, CD49d, or a combination thereof. In further aspects, the TRAM^{lo} monocytes or TRAM deficient monocytes exhibit elevated expression of resolving mediators comprising healthy mitochondria, peroxisome, CD200R, CD24, Sestrin1 (SESN1), Sestrin 2 (SESN2), Sestrin3 (SESN3), or a combination thereof. The elevated expression of anti-inflammatory resolving mediators paired with a reduced expression of inflammatory mediators can beneficially provide for compositions and methods for use in treating chronic inflammatory diseases.

In embodiments, the methods described herein regarding the enrichment of TRAM 1° neutrophils or the reprogramming of innate neutrophils into TRAM deficient neutrophils may be further applied to the methods described herein regarding the enrichment of TRAM 1° monocytes or the reprogramming of human monocytes into TRAM deficient monocytes. For example, in the disclosed methods of the disclosure, the innate neutrophils and human monocytes may be reprogrammed from a pro-inflammatory state to a resolution state caused by TRAM deficiency. In some aspects, resolving neutrophils and monocytes (i.e., neutrophils and monocytes in the resolution state) not only express less inflammatory mediators and caspases, but also express resolving mediators as described herein. In some examples, the resolving mediator RvD1 serves as an effective lipid resolving mediator capable of reducing tissue inflammation. In further examples, CD200R can suppress inter-cellular inflammatory activation, and SESN1 is a potent suppressor of oxLDL-mediated inflammasome activation and subsequent degranulation.

In some regards, TRAM is highly expressed in the majority subsets of inflammatory and apoptotic leukocytes, however, tend to be absent in the minor population of resolving leukocytes. Consequently, in some implementations, the genetic deletion of TRAM can reprogram leukocytes, such as neutrophils and monocytes, into the resolving state. In embodiments where the innate neutrophils or human monocytes are modified to result in TRAM deficient neutrophils or TRAM deficient monocytes, respectively, the innate neutrophils or human monocytes may be modified utilizing any method for the genetic reduction or deletion of TRAM. In some embodiments, the modification methods may include, but are not limited to, modification by CRISPR knockout of TRAM gene, anti-sense oligonucleotide (AS-ODN) of TRAM mRNA, proteolysis-targeting chimeras (PROTAC) degradation of TRAM protein, reduction of cellular levels of TRAM using a pharmacological treatment comprising 4-phenylbutyric acid (4-PBA), methoxy-mycolic acid, docosahexaenoic acid (DHA), or fumagillin, or a combination thereof. In some embodiments, and without being limited to any particular mechanism or theory, TRAM deficiency (or TRAM^{lo} neutrophils and monocytes) can result in the reduction of LOX5AP (FLAP) expression. Beneficially, reduction of LOX5AP can dislodge nuclear localization of LOX5, and favor the generation of resolving mediators instead of inflammatory mediators.

In embodiments, the TRAM^{lo} neutrophils, innate neutrophils, TRAM^{lo} monocytes, or human monocytes may be isolated or sorted for further therapeutic uses as further described herein. While any method of sorting or isolation may be suitable, in some aspects, the TRAM^{lo} neutrophils, TRAM deficient neutrophils, innate neutrophils, TRAM^{lo} monocytes, TRAM deficient monocytes, or human monocytes may be isolated by flow cytometry or magnetic bead-based separation. In embodiments, the TRAM^{lo} neutrophils, innate neutrophils, TRAM^{lo} monocytes, or human monocytes may be optionally supplemented or treated with low dose granulocyte-macrophage colony-stimulating factor (GM-CSF), low dose granulocyte colony-stimulating factor (G-CSF), or low dose macrophage colony-stimulating factor (M-CSF). In some aspects, the neutrophils described in the present disclosure may be optionally supplemented or treated with the GM-CSF, G-CSF, or a combination thereof. In further aspects, the monocytes described in the present disclosure may be optionally supplemented or treated with the M-CSF. In some aspects, the concentration of GM-CSF, G-CSF, or M-CSF may be present in an amount of between about 1 ng/mL to about 150 ng/mL, between about 2 ng/mL to about 130 ng/mL, between about 3 ng/mL to about 120 ng/mL, between about 4 ng/mL to about 120 ng/mL, or between about 5 ng/mL to about 100 ng/mL. In some aspects, the GM-CSF may be used to maintain cell viability during the reprogramming process. In further embodiments, the neutrophils and monocytes may be optionally treated with an additional agent comprising cell stress relievers such as, but not limited to, DHA, 4-PBA, or a combination thereof. The additional agent may be administered at a concentration of about 0.001 μg/mL to about 40 μg/mL, about 0.01 μg/mL to about 30 μg/mL, or about 0.01 μg/mL to about 20 μg/mL.

The disclosures further provide for methods of treating a chronic inflammatory disease in a subject, wherein the method comprises administering the TRAM^{lo} neutrophils, TRAM deficient neutrophils, TRAM^{lo} monocytes, or TRAM deficient monocytes as described herein to the subject. In embodiments, the TRAM^{lo} neutrophils, TRAM deficient neutrophils, TRAM^{lo} monocytes, or TRAM deficient monocytes have been enriched or reprogrammed as already described herein. In embodiments, the TRAM^{lo} neutrophils or TRAM deficient neutrophils express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof, and are isolated or sorted prior to administering to the subject in need thereof. In further embodiments, the TRAM^{lo} monocytes or the TRAM deficient monocytes express surface markers comprising CD14⁺, CD16⁺, and at least one of CD24^{hi}, CD200R^{hi}, or CD84^{hi}, and are isolated or sorted prior to administering to the subject in need thereof. In some embodiments, the subject is a mammal. In further embodiments, the mammal is a human.

In some embodiments, the immune-enhancing neutrophils may be provided as a pharmaceutical formulation, or pharmaceutical composition, that can include a pharmaceutically acceptable carrier thereof. The pharmaceutical compositions can be used to treat and/or prevent diseases, such as a chronic inflammatory disease. The disclosed neutrophils and monocytes may be provided in a dosage form. The dosage forms can be adapted for administration by any appropriate route. In embodiments, the preferred route of administration is via intravenous injection. In further embodiments, other appropriate routes can include, but are not limited to, epidural, intracranial, intraocular, vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from concentrated cell solutions, sterile powders, granules, and tablets.

For some embodiments, the dosage form contains a predetermined amount of the enriched or reprogrammed neutrophils or monocytes described herein per unit dose. In some embodiments, the predetermined amount of the enriched or reprogrammed neutrophils or monocytes described herein is a therapeutically effective amount effective to treat or prevent a chronic inflammatory disease or disorder. In other embodiments, the predetermined amount of the enriched or reprogrammed neutrophils or monocytes described herein can be an appropriate fraction of the therapeutically effective amount of the active ingredient (e.g., enriched or reprogrammed neutrophils or monocytes described herein and/or auxiliary active agent). Such pharmaceutical formulations may be prepared by any of the methods well known in the art. In some embodiments, the enriched or reprogrammed neutrophils or monocytes described herein are administered to the subject in the form of a blood transfusion via an intravenous dose of administration. The amount of blood containing the immune-enhancing neutrophils to be transferred via the blood transfusion may be in the range of between about 5 mL and about 500 mL, between about 10 mL and about 400 mL, between about 60 mL and about 390 mL, between about 70 mL and about 380 mL, between about 80 mL and about 370 mL, between about 90 mL and about 360 mL, or between about 90 mL and about 350 mL. In some embodiments, from about 50 mL to about 500 mL of blood containing the TRAM^{lo} neutrophils or TRAM deficient neutrophils is administered to the subject via the blood transfusion. In further embodiments, 10 mL to about 500 mL of blood containing the TRAM^{lo} monocytes or TRAM deficient monocytes is administered to the subject via the blood transfusion.

In embodiments, the enriched or reprogrammed neutrophils or monocytes described herein may be administered to the subject at least once weekly, at least two times per week, or at least three times per week. In some embodiments, the enriched or reprogrammed neutrophils or monocytes described herein may be administered to the subject at least once daily, or more than once per day. In other embodiments, the enriched or reprogrammed neutrophils or monocytes described herein and pharmaceutical formulations thereof can be administered one or more times per month, such as 1 to 5 times per month, such as 1, 2, 3, 4 or 5 times per month. In still further embodiments, the enriched or reprogrammed neutrophils or monocytes described herein and pharmaceutical formulations thereof can be administered one or more times per year, such as 1 to 11 times per year. In some embodiments, the enriched or reprogrammed neutrophils or monocytes described herein and pharmaceutical formulations thereof can be administered once daily, once weekly, at least once per month, at least twice per month, at least three times per month, or at least four times per month.

In some embodiments, the methods and compositions of the disclosure improve vascular integrity, reduce plaque size, or a combination thereof when administered to a subject. In aspects, the methods and compositions provided herein may be used to prevent or treat a chronic inflammatory disease or disorder. In aspects, the chronic inflammatory disease includes, but is not limited to, atherosclerosis, stroke, depression, anxiety, suicidal ideation, a cardiovascular disease, a neurological disease, or an age-related disease. In some embodiments, the age-related disease may include, but is not limited to Alzheimer's disease or Parkinson's disease. In further embodiments, the neurological disease may include, but is not limited to, epilepsy.

In optional embodiments, the enriched or reprogrammed neutrophils or monocytes described herein and pharmaceutical formulations thereof may be co-administered with a secondary agent by any convenient route. The secondary agent is a separate compound and/or pharmaceutical formulation from the enriched or reprogrammed neutrophils or monocytes described herein or pharmaceutical formulations thereof. The secondary agent can be administered simultaneously with the enriched or reprogrammed neutrophils or monocytes described herein or pharmaceutical formulations thereof. The secondary agent can be administered sequentially with the enriched or reprogrammed neutrophils or monocytes described herein or pharmaceutical formulations thereof. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Materials and Methods

Data source: The single-cell RNA sequencing (scRNAseq) data sets were deposited at Gene Expression Omnibus (GEO) with an accession number of GSE182356, the entire contents of which are incorporated by reference herein.

Mice: Wild type (WT) C57BL/6 mice, and ApoE^{-/-} mice were purchased from the Jackson Laboratory. Tram^{-/-} mouse colony was provided by Dr. Holger Eltzschig. ApoE^{-/-} Tram^{-/-} mice were obtained by crossing ApoE^{-/-} mice with Tram^{-/-} mice. All mice were maintained on a 12 h/12 h light/dark cycle at 22° C. in a pathogen-free environment. All experimental procedures were approved by the Intuitional Animal Care and Use Committee in compliance with the U.S. National Institutes of Health Guide for the Care and Use of Laboratory Animals. Both male and female mice between 8-12 weeks of age group were used for experiments, and no gender-specific effects were observed.

Primary murine neutrophil culture: Bone marrow (BM) cells were harvested from the femur and tibia of mice, and Ly6G⁺ neutrophils in the BM were sorted by flow cytometry. The neutrophils were cultured in complete RPMI 1640 medium (containing 10% fetal bovine serum (FBS), 2 mM L-glutamine, penicillin-streptomycin) supplemented with G-CSF (100 ng/mL). In some experiments, neutrophils were treated with oxLDL (10 μg/mL), or free cholesterol (10 μg/mL) for 48 hours.

Human neutrophils isolation: Peripheral blood from healthy donors were purchased from Research Blood Components LLC. Human neutrophils were isolated by dextran sedimentation and FACS (fluorescence-activated cell sorting). Whole blood was mixed with 3% Dextran at a 1:1 ratio for 30 min at room temperature. The upper layer containing leukocytes were harvested, and residual red blood cells were removed by ACK lysis buffer. The leukocytes were blocked with Fc blocker in FACS buffer (2% FBS in 1×HBSS) for 15 minutes, and then labeled with fluorescent-conjugated anti-CD66b and anti-CD177 antibodies for 30 minutes. Propidium iodide (PI) was added to separate live and dead cells. PI-CD66b⁺ CD177^{lo} and PI⁻ CD66b⁺CD177^{hi} were sorted by Sony cell sorter SH800. Purified neutrophils were resuspended in RPMI 1640 complete medium for further use.

Cell Lines: Three different endothelial cell lines were used in this study. HUVECs and bEnd.3 cells were purchased from ATCC; immortalized mouse cardiac endothelial cells (MCECs) were provided by Dr. Tianqing Peng at the University of Western Ontario. All of the endothelial cell lines were cultured in DMEM with 10% FBS and penicillin-streptomycin.

HFD Feeding and Neutrophil Adoptive Transfer: Age and gender matched recipient ApoE^{-/-} Tram^{+/+} mice were fed with a high-fat diet (HFD) for 4 weeks to induce the development of atherosclerosis. Ly6G⁺ neutrophils were sorted from the BM of ApoE^{-/-} Tram^{-/-} mice or control ApoE^{-/-} Tram^{+/+} mice using flow cytometry. Adoptive transfer of neutrophils was conducted. Each HFD-fed recipient mouse was transfused with 2×10⁶ neutrophils in 200 μl of PBS once weekly through intravenous injection for a total of 4 injections. The mice were continuously maintained on HFD throughout the adoptive transfer regimen. One week after the final cell transfer, the mice were sacrificed, and tissues were harvested for subsequent analyses.

Histological Analysis of Atherosclerotic Lesions: Histological analyses of atherosclerotic lesions were performed as previously described. Fresh-frozen and optimal cutting temperature (OCT) compound-embedded proximal aortic sections (10 μm) were fixed in 4% neutral buffered formalin followed by H&E staining. Oil Red O staining was performed using a kit (Newcomer Supply), and collagen staining was performed using the Picrosirius Red Stain Kit (Polysciences) according to the manufacturers' instructions. The samples were mounted with Canada balsam or Permount mounting medium and observed under a light microscope. Quantification of the samples was performed with ImageJ. The percentages of total lesion area, lipid deposition, and collagen composition were calculated.

ELISA: Plasma samples were collected from the mice that received neutrophil transfer. The levels of RvD1, elastase, LTB4, and MPO were determined with commercially available ELISA kits. For the analysis of RvD1 and elastase levels in cell culture supernatant, Ly6G⁺ neutrophils were sorted from the BM of Tram^{-/-} mice and WT mice and then treated with oxLDL (10 μg/mL) for 48 hours. The resulting supernatant was collected with the quantification of RvD1 and elastase. Sorted human neutrophils were plated into 96-well plates, and stimulated with or without fMLP (100 nM) for 4 hours, then supernatant was collected to determine LTB4 and elastase levels. All the assays were conducted following the manufacturers' instructions. Detailed information of the ELISA kits can be found in the Key Resources Table.

Assessment of Aortic Endothelium Permeability: The aortic endothelium permeability was determined using an established protocol with modifications. ApoE^{-/-} Tram^{+/+} mice were transfused with ApoE^{-/-} Tram^{-/-} or control ApoE^{-/-} Tram^{+/+} neutrophils as described above. One week post the final cell transfer, 200 μL of 0.5% Evans Blue solution (in PBS) was intravenously injected to each recipient mouse. After 1 hour, the mouse was sacrificed, and the blood and aorta were collected. The serum was prepared and preserved at −20° C. The aorta was rinsed with PBS, weighted, and put in a conical microfuge tube containing 250 μL of formamide, followed by incubation at 55° C. for 48 hours. The aorta sample was centrifugated, and the supernatant was collected. The absorbance of serum and aorta supernatant samples, as well as Evans Blue standards was measured at 620 nm. The Evans Blue concentrations in the serum and aorta samples were calculated. The Evans Blue concentration of aorta sample was divided by aorta weight and then by serum Evans Blue concentration for normalization.

Flow Cytometry Analysis of Aortic Endothelial Cells: ApoE^{-/-} Tram^{+/+} mice were transfused with ApoE^{-/-} Tram^{-/-} or ApoE^{-/-} Tram^{+/+} neutrophils as described above. One week post the final neutrophil transfer, aortas were harvested and single-cell suspensions were prepared for flow cytometry as previously reported. Aortas were cut into small pieces and then transferred into an enzyme cocktail (in HBSS) containing 450 U/ml collagenase type I, 250 U/ml collagenase type XI, 120 U/ml hyaluronidase, and 120 U/ml DNAse supplemented with 20 mM HEPES. The samples were incubated at 37° C. for 60 min. The processed samples of all tissues were filtered through 70-μm cell strainers to obtain single cell suspension, and red blood cells were lysed with ACK buffer. The samples were incubated with anti-CD16/-CD32 antibody to block Fc-receptors followed by staining with fluorochrome-conjugated anti-CD45, anti-CD31 and anti-VCAM-1 antibodies. PI was added before flow cytometry to label dead cells. The expression of VCAM-1 on the surface of PI⁻ CD45⁻ CD31⁺ endothelial cells was examined using FACSCanto II (BD Biosciences). The data were analyzed using FlowJo.

Neutrophil-endothelial Cell Co-Culture, Transwell-Evans Blue Permeability Assay, and Flow Cytometry Analysis: Ly6G⁺ neutrophils were sorted from the BM of Tram^{-/-} mice and WT mice. To determine the effect of neutrophils on the integrity of endothelial cell layer, Transwell-Evans Blue permeability assay were conducted according to previously established protocol with modifications. Briefly, bEnd.3 cells or MCECs were seeded to each Transwell insert (0.4 μm pore size) for 12-well plates and cultured for 24 hours to form a monolayer. Tram^{-/-} or WT neutrophils were added to the insert at a 10:1 ratio (neutrophils to endothelial cells), followed by co-culture for 24 hours. The medium in the insert was discarded, the insert was transferred to a new 12-well plate containing 4% BSA, and 400 μL of Evans Blue solution (0.67 ng/mL) was added to the insert. After incubation for 30 minutes, the absorbance of the medium in the lower chamber of the Transwell system was measured at 620 nm, allowing for the quantification of Evans Blue leakage. Evans Blue leakage to the lower chamber of Transwell system that only contained with endothelial cell monolayer without adding neutrophils was employed for normalization. To examine the expression of VCAM-1 on endothelial cells, HUVECs, bEnd.3 cells or MCECs were seeded to 12-well plates and cultured for 24 hours to form a monolayer. Tram^{-/-} or WT neutrophils were co-cultured with endothelial cells at a 10:1 ratio. After 24 hours, cells were harvested, blocked with anti-CD16/-CD32 antibody, and stained with fluorochrome-conjugated anti-LyG and anti-VCAM-1 antibodies. PI was added before flow cytometry. The expression of VCAM-1 on the surface of PI⁻ Ly6G⁻ endothelial cells was examined using FACSCanto II (BD Biosciences). The death of endothelial cells was also assessed by flow cytometry based on PI staining.

Immunoblotting: Ly6G⁺ neutrophils were sorted from the BM of Tram^{-/-} mice and WT mice and then treated with oxLDL (10 μg/mL) for 48 hours. Total proteins were extracted with RIPA buffer containing a protease inhibitor cocktail. The same amount of protein samples were subjected to SDS-PAGE and transferred to a polyvinylidene difluoride membrane, which was then incubated with blocker at room temperature for 1 hour. The membrane was incubated with primary anti-FLAP, anti-SESN1 or β-actin antibody overnight at 4° C., followed by incubation with horseradish peroxidase (HRP)-conjugated anti-rabbit IgG for 1 hour at room temperature. Blots were developed by a chemiluminescence ECL detection kit.

Confocal microscopy: Ly6G⁺ neutrophils were sorted from the BM of Tram^{-/-} mice and WT mice and then treated with oxLDL (10 μg/mL) or free cholesterol (10 μg/mL) for 48 hours. Peroxisome-lysosome fusion in neutrophils was examined by confocal microscopy with a protocol. Neutrophils were fixed with 4% paraformaldehyde, deposited on slides by cytospin, and permeabilized with 0.2% Triton X-100. The samples were then blocked and stained with primary rabbit anti-mouse PMP70 antibody followed by staining with Alexa Fluor 488-conjugated goat anti-rabbit secondary antibody to label peroxisomes. The blocking solution and antibodies were supplied in the SelectFX Alexa Fluor 488 Peroxisome Labeling Kit. After extensive washing with PBS, the cells were then stained with Cy3-conjugated anti-LAMP1 antibody to label lysosomes. To detect lysosomal distribution of CAMKII and SYK, peroxisomes in the neutrophils were first labeled as described above. After washing with PBS, the neutrophils were stained with Alexa Fluor 546-conjugated anti-CAMKII antibody or PE conjugated anti-SYK antibody. To observe the intracellular distribution of LOX5 and TRAM, neutrophils were fixed, deposited on slides, permeabilizated, and blocked with Streptavidin/Biotin Blocking Solution. The samples were then stained with biotin-conjugated anti-LOX5 antibody or biotin-conjugated anti-TRAM antibody, followed by Cy3-conjugated streptavidin or Alexa Fluor 488-conjugated streptavidin, respectively. The nuclei were counterstained with DAPI. All the samples were mounted with antifade mountant and observed under LSM 900 confocal microscope (ZEISS). Images were processed with ZEN lite.

Detection of Intracellular Reactive Oxygen Species (ROS): Ly6G⁺ neutrophils were sorted from the BM of Tram^{-/-} mice and WT mice and then treated with oxLDL (10 μg/mL) for 48 hours. Intracellular ROS level was determined, and CellROX Green Reagent was added to neutrophil cultures 30 min before harvesting. The samples were examined using FACSCanto II, and the data were analyzed using FlowJo.

Flow Cytometry Analyses of Human Neutrophils: Peripheral blood from healthy individual was purchased from Research Blood Components LLC. After lysing red blood cells, the samples were blocked with Fc Receptor Blocking Solution, and stained with FITC-conjugated anti-CD66b and PE-conjugated anti-CD177 antibodies. The cells were then fixed and permeabilized using Cyto-Fast™ Fix/Perm Buffer, followed by staining with Alexa Fluor 647-conjugated anti-TRAM antibody. The expression of CD177 and TRAM in CD66b⁺ neutrophils was examined using FACSCanto II, and the data were analyzed using FlowJo.

Swarming Assay: The slides were pre-coated with inactivated-fungi, and assembled with multi-well attachment. CD177^{lo} and CD177^{hi} human neutrophils were sorted and fluorescent labeled. Specifically, CD177^{lo} neutrophils were labeled with Hoechst 33342 dye at final concentration of 10 μM and CD177^{hi} neutrophils were labeled with DRAQ5 dye at final concentration of 10 μM for 15 min at 37° C. After washed, neutrophils were resuspended in RPMI complete medium at the concentration of 2.5 million cells per ml, then 200 μl were added to each well. Time-lapse images were recorded by KEYENCE fluorescence microscope. Quantification of the images was performed with ImageJ.

Quantification and Statistical Analysis: Statistical analyses were conducted using Prism software. All data are expressed as means±SEM. Comparisons between two groups were performed with using 2-tailed Student's t test, and comparisons among multiple groups were carried out with one-way ANOVA. P<0.05 was considered statistically significant.

### Example 2

### Transfusion of Tram^{-/-} Neutrophils for Potently Alleviating the Pathogenesis of Atherosclerosis

The effects of Tram^{-/-} neutrophils for use in therapeutically treating experimental atherosclerosis were analyzed. To test this, neutrophil transfusion studies were conducted with atherosclerosis-prone recipient mice. Both male and female ApoE^{-/-} Tram^{+/+} mice were used as recipient mice, which were initially fed with a high-fat diet (HFD) for 4 weeks. The mice subsequently received intravenous injection of neutrophils purified from the bone marrow (BM) of either ApoE^{-/-} Tram^{-/-} mice or control ApoE^{-/-} Tram^{+/+} mice. The adoptive transfer of neutrophils were performed weekly for 4 weeks, and the recipient mice were all supplemented with HFD during the entire process of the experiment, allowing the development of atherosclerosis. The mice were sacrificed for analysis one week after the final neutrophil injection.

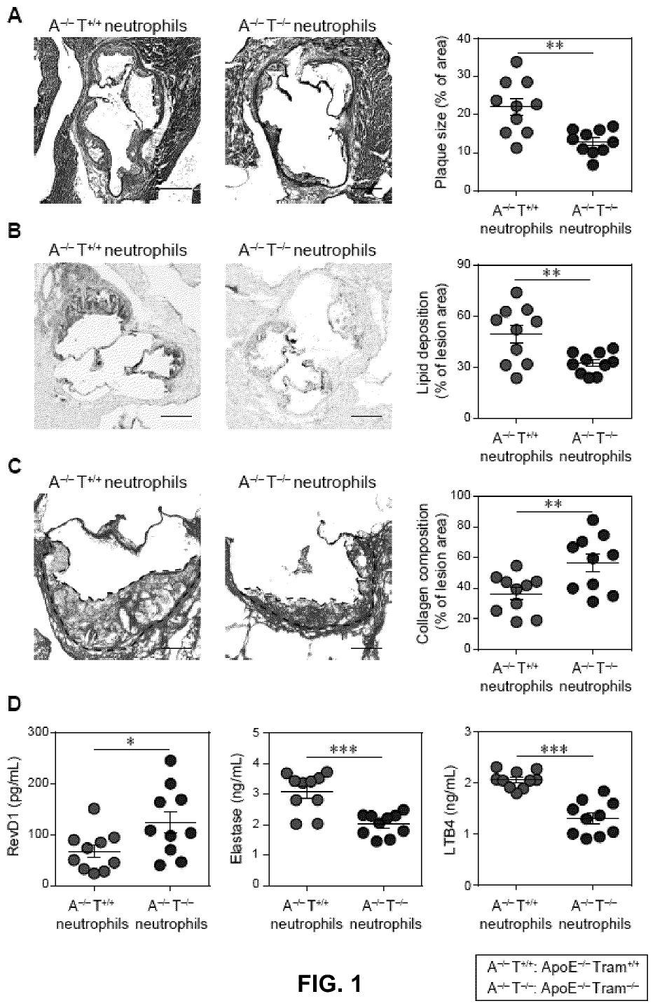

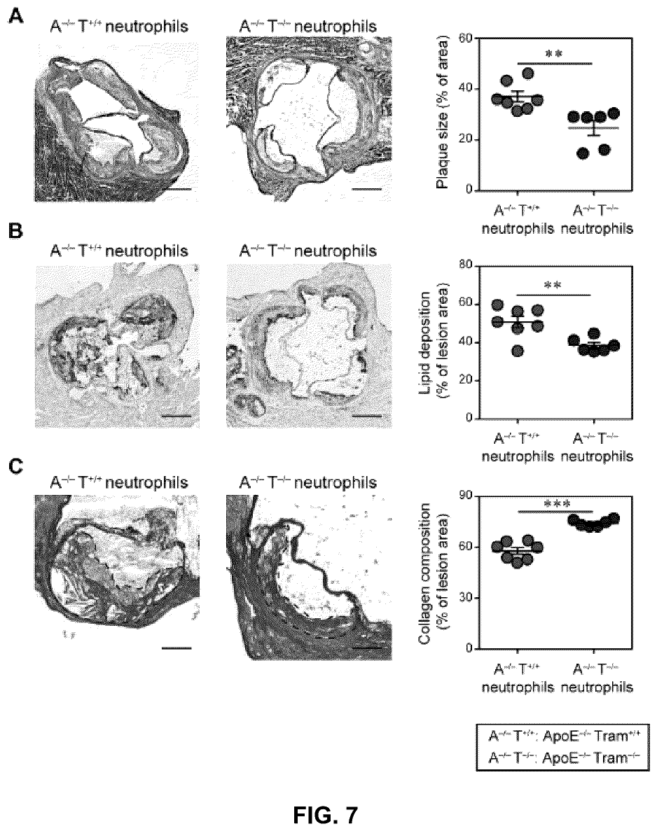

Histological assessments through hematoxylin and eosin (H&E) staining and Oil-Red O staining revealed that both male and female mice receiving the transfer of Tram^{-/-} neutrophils had significant reduction in plaque sizes (FIGS. 1A and 7A) as well as remarkably decreased lipid deposition area within atherosclerotic lesions (FIGS. 1B and 7B) as compared to the counterparts receiving Tram^{+/+} neutrophils. Picrosirius Red staining demonstrated a significantly elevated level of collagen content in plaques following the adoptive transfer of ApoE^{-/-} Tram^{-/-} neutrophils in comparison to ApoE^{-/-} Tram^{+/+} neutrophils (FIGS. 1C and 7C).

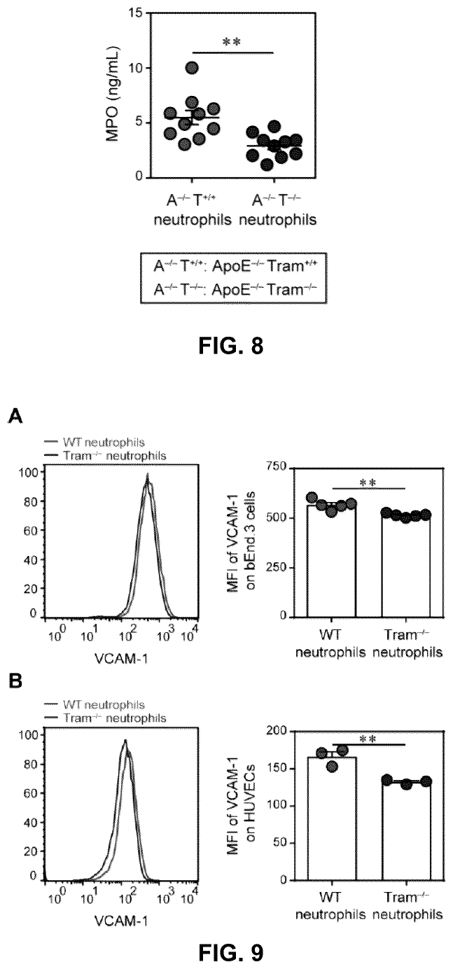

The plasma levels of key mediators produced by neutrophils were further examined. Notably, adoptive transfer of ApoE^{-/-} Tram^{-/-} neutrophils led to a significant elevation of resolving product RvD1, accompanied by a significant decrease in pro-inflammatory mediators such as LTB4, elastase and MPO (FIGS. 1D and 8). The data indicates that adoptive transfer of Tram^{-/-} neutrophils can effectively mitigate atherosclerosis progression and strengthen the stability of atherosclerotic plaques, and this approach may serve as an avenue for immune cell therapy in the treatment of atherosclerosis. These observed effects may be associated with the alteration of neutrophil functionalities from a pro-inflammatory state to a resolution state caused by TRAM deficiency. Since there was no gender disparity in the effectiveness of neutrophil transfusion, only male ApoE^{-/-} Tram^{+/+} mice were used as recipients in the following in vivo study Examples.

### Example 3

### Transfusion of Tram^{-/-} Neutrophils for Effectively Improving Vasculature Integrity

The disruption in the function and integrity of the arterial endothelial cell can crucially contribute to the development of atherosclerosis. Therefore, the effects of Tram^{-/-} neutrophils on vasculature integrity was analyzed. Neutrophil elastase has been identified as a factor that increases the permeability of endothelial cells, while RvD1 exerts a robust protective effect in enhancing endothelial integrity and barrier function. As discovered in Example 2, Trami-neutrophils exhibited elevated levels of RvD1 and reduced secretion of elastase. Further, in vivo adoptive transfer data revealed elevated plasma levels of RvD1 and reduced elastase in recipient mice receiving Tram^{-/-} neutrophils.

To evaluate whether Tram^{-/-} neutrophils may improve vascular integrity, neutrophil transfusion with the same regimen as described in Example 2 was conducted. One week post the final cell transfer, 0.5% Evans Blue solution was intravenously injected to each mouse, and the amount of Evans Blue leaked from the circulation into the aorta was measured. In a separate transfusion experiment, the expression of VCAM-1, a pro-inflammatory molecule crucial for atherosclerosis development, on the surface of aortic endothelial cells was assessed.

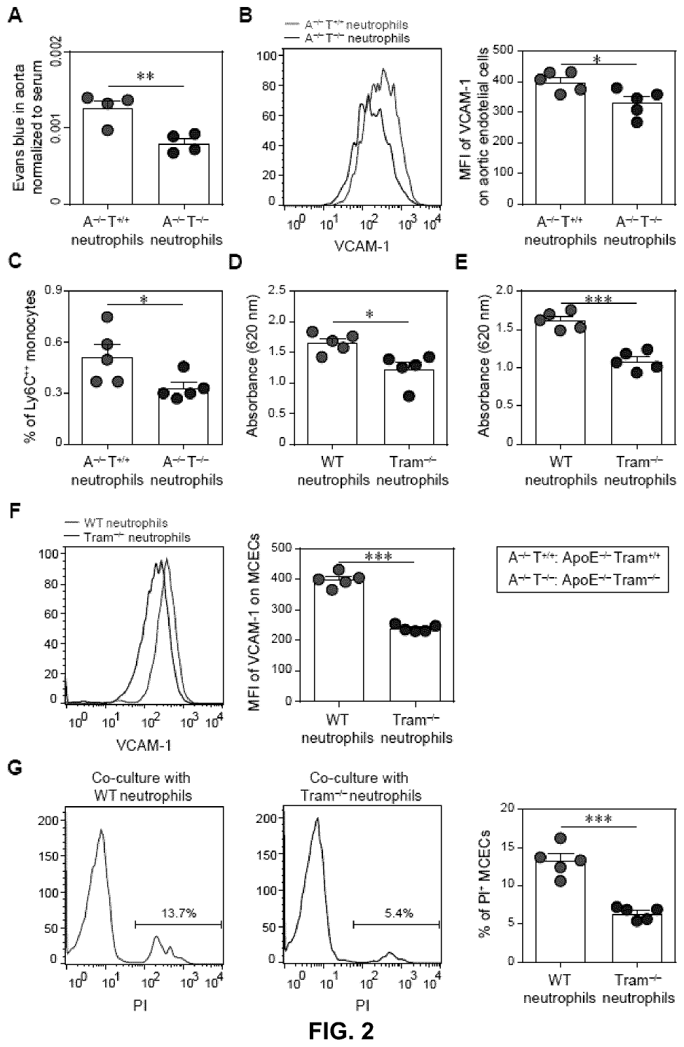

The results demonstrated that the atherosclerotic mice receiving the transfusion of ApoE^{-/-} Tram^{-/-} neutrophils had significantly reduced Evans Blue accumulated in the aorta as compared with the mice receiving ApoE^{-/-} Tram^{+/+} neutrophils (FIG. 2A). Further, flow cytometry analysis revealed that adoptive transfer of ApoE^{-/-} Tram^{-/-} neutrophils dramatically decreased VCAM-1 expression on the endothelial cells (FIG. 2B), accompanied by a notable reduction of Ly6C⁺⁺ pro-inflammatory monocyte infiltration into the aorta (FIG. 2C). These data demonstrated that transfusion of TRAM-deficient neutrophils effectively enhanced the vascular endothelial integrity and also alleviated the pro-inflammatory state of endothelial cells in atherosclerotic mice.

Within this Example, the protective effects of Tram^{-/-} neutrophils on endothelial cells with an in vitro Transwell-Evans Blue permeability assay was assessed. In this assessment, bEnd.3 cells, an endothelial cell line derived from the mouse brain, or immortalized mouse cardiac endothelial cells (MCECs) were seeded into the inserts to form a monolayer, which was then co-cultured with neutrophils purified from wild type (WT) or Tram^{-/-} mice. The permeability of the endothelial cell monolayer was determined by measuring the leakage of Evans Blue from the upper chamber to the lower chamber.

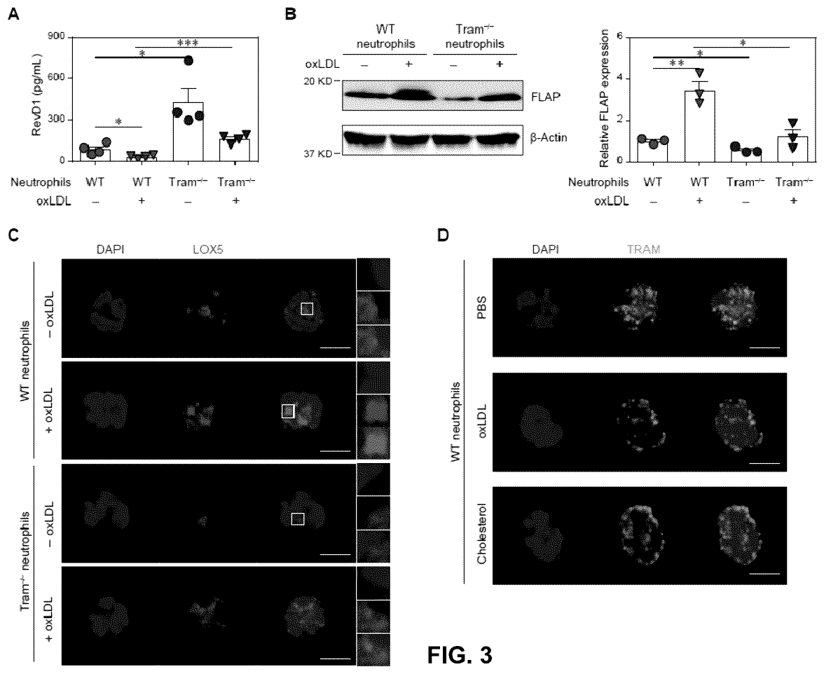

The result demonstrated a significant reduction in Evans Blue amounts in the lower chambers when endothelial cells were co-cultured with Tram^{-/-} neutrophils compared to those co-cultured with WT neutrophils (FIGS. 3D and 3E), indicating that TRAM deficiency in neutrophils substantially enhanced the integrity of both bEnd.3 and MCEC layers. Furthermore, in line with in vivo observations, co-culture of Tram^{-/-} neutrophils led to a significantly decreased expression of VCAM-1 on the surface of diverse endothelial cell types, including MCECs (FIG. 3F), bEnd.3 cells (FIG. 9A), as well as human umbilical vein endothelial cells (HUVECs) (FIG. 9B). The death of endothelial cells is a pivotal initiating event in atherogenesis, and atherosclerotic plaques contain abundant apoptotic endothelial cells. Given this context, Tram^{-/-} neutrophils may exert a protective effect by attenuating the death of cardiac endothelial cells. Indeed, flow cytometry analysis unveiled a significantly higher viability of MCECs when co-cultured with Tram^{-/-} neutrophils compared to those co-cultured with WT neutrophils (FIG. 3G).

These results demonstrated the critical role of TRAM expressed by neutrophils in modulating endothelial cells toward an atherosclerosis-prone state. Therefore, the results suggest that TRAM deficiency may be used as a therapeutic target for preventing and treating atherosclerosis.

### Example 4

### Tram Deletion on FLAP Expression and Effects of LOX5

As identified in Example 2, elevated levels of RvD1 were observed in the circulation of atherosclerotic mice post-transfusion of Tram^{-/-} neutrophils. Therefore, it is considered that Tram^{-/-} neutrophils may represent an important source of RvD1. Incubation with oxLDL remarkably attenuated RvD1 production by WT neutrophils, while conversely, Tram-neutrophils exhibited resistance to oxLDL-mediated suppression of RvD1 production.

Moreover, the baseline RvD1 production of Tram^{-/-} neutrophils surpassed that of their WT counterparts, suggesting that TRAM functions as a negative modulator of RvD1 production in neutrophils and the absence of TRAM leads to constitutive production of high levels of RvD1 (FIG. 3A). Therefore, in this Example, key molecular mechanisms involved in the elevated generation of RvD1 in Tram^{-/-} neutrophils were evaluated.

Through previous scRNAseq data generated, it was observed that a drastically reduced expression of Lox5ap (FLAP), a key molecule responsible for anchoring LOX5 onto the nuclear membrane and enabling the production of inflammatory mediator LTB4, was observed. On the other hand, when 5-lipoxygenase (LOX5) was not anchored on the nuclear membrane, cytosolic LOX5 was channeled into generating the resolving lipid mediator RvD1. In this Example, through immuno-blot analyses, it was observed that Tram^{-/-} neutrophils expressed significantly less FLAP protein as compared to WT neutrophils under steady-state conditions. Upon priming with oxLDL, a substantial elevation in FLAP protein expression was observed in WT neutrophils, while in contrast, oxLDL-induced FLAP increase was significantly attenuated in Tram^{-/-} neutrophils (FIG. 3B).

Confocal microscopy was further conducted to examine the subcellular distribution of LOX5. WT neutrophils preferentially expressed LOX5 within the nuclear membrane area, and oxLDL treatment elevated the nuclear membrane associated LOX5 protein. In sharp contrast, it was observed that TRAM deletion disrupted the nuclear localization of LOX5 (FIG. 3C).

The results of the confocal microscopy examination yielded compelling evidence for TRAM serving as a general membrane stress sensor for oxLDL or free cholesterol in addition to TLR ligands. The results demonstrated that either oxLDL or free cholesterol treatment induced a remarkable translocation of TRAM in neutrophils and promoted TRAM clustering on the cell membrane (FIG. 3D). These data unveiled the role of TRAM as a sensor for diverse lipid-induced membrane stress, which induces the translocation of LOX5 from cytosol to the nucleus and subsequently suppresses RvD1 production. Therefore, TRAM deletion can reverse these events, restoring RvD1 production and facilitating inflammation resolution.

### Example 5

### Tram Deletion on Elevated Expression of SESN1, Reduced SYK, CAMKII, and Reduced Expression of Elastase

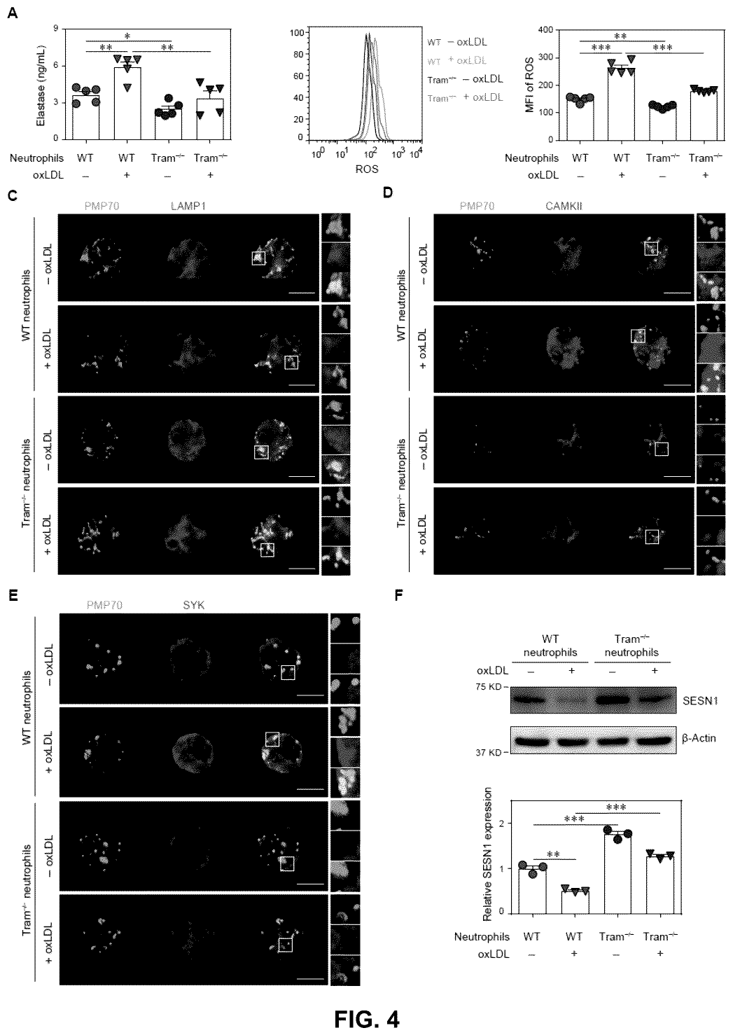

As demonstrated in previous Examples of the present disclosure, in vivo data revealed that adoptive transfer of Tram^{-/-} neutrophils led to decreased elastase in atherosclerotic mice. It has been observed that the elastase release from Tram^{-/-} neutrophils was significantly diminished as compared to that from WT neutrophils, both with and without oxLDL treatment (FIG. 4A). Therefore, in this Example, the molecular mechanisms responsible for the reduced secretion of elastase from Tram^{-/-} neutrophils was examined.

Secretion of neutrophil elastase can be mediated by the generation of cellular ROS. As also described in Example 1, the assessment of cellular ROS revealed that oxLDL induced ROS generation in WT neutrophils, and TRAM deletion attenuated the ROS induction (FIG. 4B). In previous studies, it has been identified that disrupted peroxisome homeostasis is a key trigger for inflammatory signaling and intracellular ROS accumulation. Additionally, TRAM has been identified to have the capability to transmit the signal of super-low dose LPS, leading to peroxisomal dysfunction. As TRAM may function as a sensor for lipid-induced stress, confocal imaging analyses of pexophagy in neutrophils treated with oxLDL was performed in this Example.

In WT neutrophils, oxLDL treatment drastically disrupted the fusion of peroxisomes (PMP70⁺) and lysosomes (LAMP1⁺). Tram^{-/-} neutrophils exhibited constitutively elevated peroxisome-lysosome fusion, and this fusion was minimally affected by oxLDL (FIG. 4C). Activation of calmodulin-dependent kinase II (CaMKII) by metabolic stress catalyzes the transformation of glucose into ROS. It was observed that oxLDL potently increased the localization of CaMKII at peroxisomes in WT neutrophils (FIG. 4D). Further, oxLDL increased the peroxisomal distribution of the SRC kinase SYK (FIG. 4E), which was shown to form a mutually activating positive feedback loop with subcellular ROS. These findings align with previous studies, demonstrating the crucial role of peroxisomes as a platform for sustaining the inflammatory signaling cascade.

Importantly, oxLDL failed to induce SYK and CAMKII aggregation around peroxisomes in TRAM deficient neutrophils (FIGS. 4D and 4E). Collectively, the data reveals that TRAM is responsible for oxLDL-mediated disruption of pexophagy and induction of inflammatory signaling processes responsible for the neutrophil release of elastase.

In addition to ROS, inflammasome activation also triggered the release of neutrophil granules. It was identified from previous scRNAseq data that TRAM deficient neutrophils had elevated expression of SESN1, a key homeostatic molecule capable of suppressing inflammasome activation. Therefore, in this Example, an immuno-blot assay was further performed and validated the increased expression of SESN1 in TRAM deficient neutrophils as compared to WT neutrophils. Moreover, oxLDL treatment remarkably increased SESN1 expression in WT neutrophils but had minimal impact on Tram^{-/-} neutrophils in terms of SESN1 level (FIG. 4F). Therefore, the data suggest that reduced peroxisome-associated inflammatory signaling coupled with elevated homeostatic mediator SESN1 may collectively lead to reduced elastase secretion in TRAM deficient neutrophils.

### Example 6

### Phenotype of Human Neutrophil Subset with Reduced TRAM Expression

To examine human relevance, ex vivo testing of human neutrophils were further evaluated. Fresh neutrophils from healthy human donors were segregated into CD177^{hi} and CD177^{lo} populations, consistent with scRNAseq data conserved from mice to humans. The neutrophils were co-stained with a TRAM specific antibody. The functional relevance of human neutrophils with reduced TRAM expression were further evaluated. Since inflammatory neutrophils with higher expression of LTB4 and CD11b tend to exhibit exacerbated swarming ability, the swarming potential of sorted human neutrophils were further analyzed.

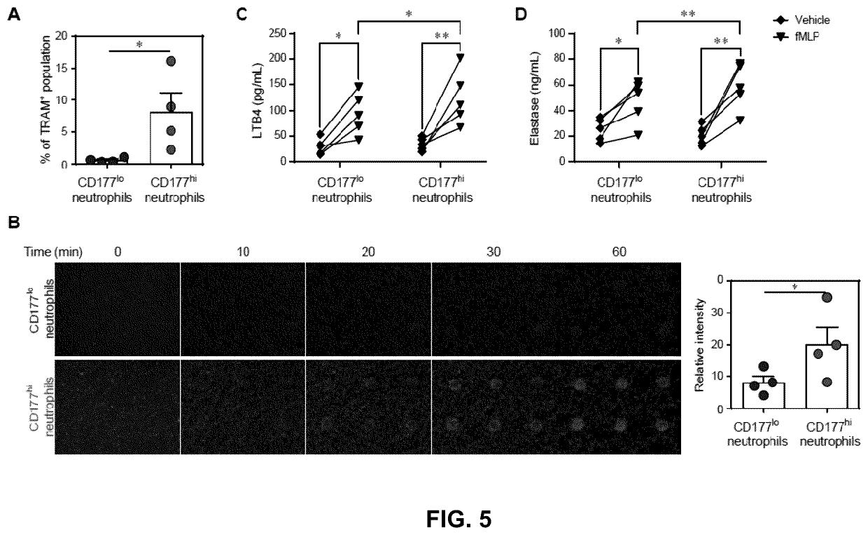

It was observed that TRAM was preferentially expressed in CD177^{hi} neutrophils and was largely absent from the CD177^{lo} neutrophils (FIG. 5A). These results were consistent with the findings with murine neutrophils in previous Examples within the present disclosure. With regard to the swarming potential of the sorted human neutrophils, as shown in FIG. 5, human CD177^{lo} neutrophils have significantly reduced swarming behaviors as compared to CD177^{hi} neutrophils (FIG. 5B).

Human neutrophils were further subject to ex vivo priming with fMLP to elicit the secretion of pro-inflammatory mediators, such as LTB4 and elastase. Both human CD177^{lo} and CD177^{hi} neutrophils were capable of secreting LTB4 and elastase in response to fMLP treatment. However, human CD177^{lo} neutrophils secreted significantly less LTB4 and elastase as compared to CD177^{hi} neutrophils, consistent with the observation of reduced swarming ability of CD177^{lo} neutrophils (FIGS. 5C and 5D).

### Example 7

### Identification of Neutrophil Subset with Anti-Inflammatory Functions

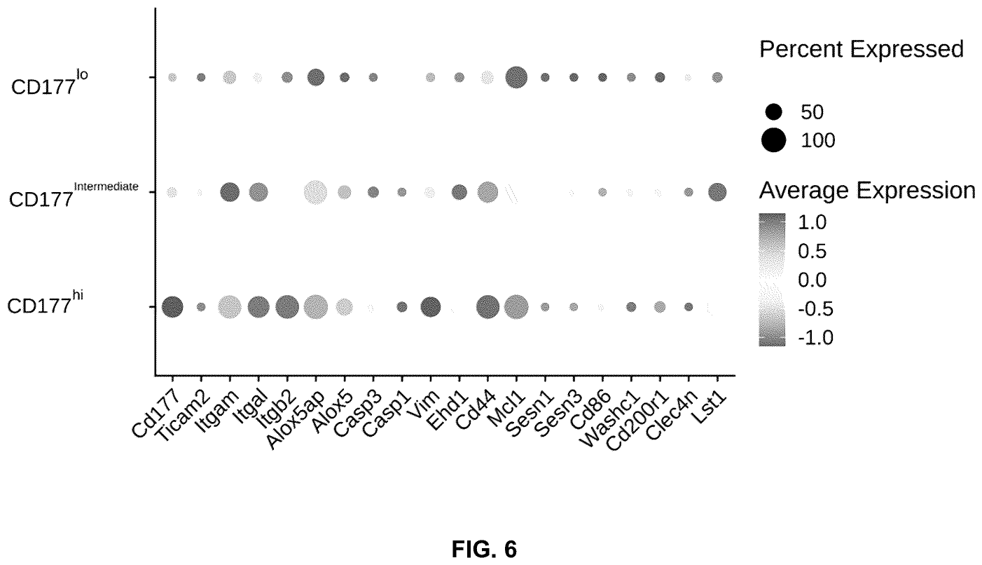

Single cell RNA sequencing (scRNAseq) analysis was completed on murine neutrophils to identify subsets of neutrophils with anti-inflammatory functions capable of reducing inflammation and improving cardiovascular function, the scRNAseq data sets of which were deposited at Gene Expression Omnibus (GEO) with an accession number of GSE182356 as identified in Example 1. Purified naïve mouse bone marrow were cultured in the presence of G-CSF (100 ng/ml) to perform the single cell sequencing. Differentially expressed genes were identified in the bubble plot shown in FIG. 6.

A particular subset of neutrophils with anti-inflammatory functions capable of reducing inflammation and improving cardiovascular function were identified from the scRNAseq data set. Therefore, in combination with the results shown in FIG. 6, it was identified that the subset of neutrophils with anti-inflammatory functions could be sorted by flow cytometry based on a unique combination of cell surface markers, CD177^{lo}/EHD1^{lo}, Ly6A/Ly6E^{hi}, and/or Dectin2 (Clec4n)^{lo}.

The above specification provides a description of the manufacture and use of the disclosed compositions and methods. Since many embodiments can be made without departing from the spirit and scope of the disclosure, the disclosure resides in the claims.

What is claimed is:

1. A method of enriching TRAM^{lo} neutrophils, or reprogramming innate neutrophils into TRAM deficient neutrophils, the method comprising:
receiving innate neutrophils isolated from a heterologous or allogenic donor; and
sorting the innate neutrophils to result in TRAM^{lo} neutrophils or modifying the innate neutrophils to result in TRAM deficient neutrophils,
wherein the TRAM^{lo} neutrophils or TRAM deficient neutrophils express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof.

2. The method of claim 1, wherein the innate neutrophils are reprogrammed from a pro-inflammatory state to a resolution state caused by TRAM deficiency.

3. The method of claim 1, wherein the surface markers comprise CD177^{lo} and Dectin 2 (Clec4n)^{lo}, or wherein the surface markers comprise CD177^{lo}, CD84^{hi}, Ly6E/Ly6A^{hi}, CD24^{hi}, and EHD1^{lo}.

4. The method of claim 1, wherein the surface markers comprise CD177^{lo}, Dectin2 (Clec4n)^{lo}, and EHD1^{lo}, and optionally one or more of CD84^{hi}, Ly6E/Ly6A^{hi}, CD24^{hi}, or CD200R^{hi}.

5. The method of claim 1, wherein the innate neutrophils are modified by CRISPR knockout of TRAM gene, anti-sense oligonucleotide (AS-ODN) of TRAM mRNA, proteolysis-targeting chimeras (PROTAC) degradation of TRAM protein, reducing cellular levels of TRAM using a pharmacological treatment comprising 4-phenylbutyric acid (4-PBA), methoxy-mycolic acid, docosahexaenoic acid (DHA), or fumagillin, or a combination thereof.

6. The method of claim 1, wherein the TRAM^{lo} neutrophils or TRAM deficient neutrophils result in the reduction of LOX5AP (FLAP) expression.

7. The method of claim 1, wherein the TRAM^{lo} neutrophils or TRAM deficient neutrophils exhibit reduced expression and/or secretion of inflammatory mediators comprising LTB4, elastase, MPO, CD11b, CD49d, or a combination thereof.

8. The method of claim 1, wherein the TRAM^{lo} neutrophils or TRAM deficient neutrophils exhibit elevated expression and/or secretion of resolving mediators comprising RvD1, CD200R, CD24, CD84, Sestrin1 (SESN1), Sestrin2 (SESN2), Sestrin3 (SESN3), CD84, Ly6E/Ly6A, MCL1, or a combination thereof.

9. The method of claim 1, further comprising a step of sorting the TRAM deficient neutrophils expressing surface markers comprising the CD177^{lo} and the at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof by flow cytometry or magnetic bead-based separation.

10. The method of claim 1, wherein the TRAM^{lo} neutrophils or innate neutrophils are isolated from human peripheral blood, and wherein the TRAM^{lo} neutrophils or innate neutrophils are further treated with low dose granulocyte-macrophage colony-stimulating factor (GM-CSF) or granulocyte colony-stimulating factor (G-CSF).

11. The method of claim 10, wherein the concentration of low dose GM-CSF or G-CSF is in a range of from about 5 ng/mL to about 100 ng/mL.

12. A method of enriching TRAM^{lo} monocytes, or reprogramming human monocytes into TRAM deficient monocytes, the method comprising:
receiving human monocytes isolated from a heterologous or allogenic donor; and
sorting TRAM^{lo} monocytes comprising TRAM^{lo} intermediate monocytes or TRAM^{lo} non-canonical monocytes, or modifying the human monocytes to result in TRAM deficient monocytes,
wherein the TRAM^{lo} monocytes or the TRAM deficient monocytes express surface markers comprising CD14⁺, CD16⁺, and at least one of CD24^{hi}, CD200R^{hi}, or CD84^{hi}.

13. The method of claim 12, wherein the human monocytes are reprogrammed from a pro-inflammatory state to a resolution state caused by TRAM deficiency.

14. The method of claim 12, wherein the human monocytes are modified by CRISPR knockout of TRAM gene, anti-sense oligonucleotide (AS-ODN) of TRAM mRNA, proteolysis-targeting chimeras (PROTAC) degradation of TRAM protein, reducing cellular levels of TRAM using a pharmacological treatment comprising 4-phenylbutyric acid (4-PBA), methoxy-mycolic acid, docosahexaenoic acid (DHA), or fumagillin, or a combination thereof.

15. The method of claim 12, wherein the TRAM^{lo} monocytes or the TRAM deficient monocytes exhibit elevated expression of resolving mediators comprising healthy mitochondria, peroxisome, CD200R, CD24, Sestrin1 (SESN1), Sestrin 2 (SESN2), Sestrin3 (SESN3), or a combination thereof, and wherein the TRAM^{lo} monocytes or the TRAM deficient monocytes exhibit reduced expression of inflammatory mediators comprising LTB4, elastase, MPO, CD11b, CD49d, or a combination thereof.

16. The method of claim 12, wherein the TRAM^{lo} monocytes or human monocytes are isolated from human peripheral blood, and wherein the TRAM^{lo} monocytes or human monocytes are further treated with low dose macrophage colony-stimulating factor (M-CSF).

17. The method of claim 16, wherein the concentration of low dose M-CSF is in a range of from about 5 ng/mL to about 100 ng/mL.

18. A method of treating a chronic inflammatory disease in a subject, the method comprising:
administering TRAM^{lo} neutrophils or TRAM deficient neutrophils to the subject, wherein the TRAM^{lo} neutrophils or TRAM deficient neutrophils express surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof.

19. The method of claim 18, wherein the TRAM^{lo} neutrophils or TRAM deficient neutrophils expressing the surface markers comprising CD177^{lo} and at least one of Dectin2 (Clec4n)^{lo}, EHD1^{lo}, CD84^{hi}, Ly6E/Ly6A^{hi}, CD200R^{hi}, CD24^{hi}, CD49D^{lo}, or a combination thereof, are sorted by flow cytometry or magnetic bead-based separation prior to administering to the subject.

20. The method of claim 18, wherein the TRAM^{lo} neutrophils or TRAM deficient neutrophils improve vascular integrity, reduce plaque size, or a combination thereof.
